(19) United States
(12) Patent Application Publication
(10) Pub. No.: US 2012/0152249 A1
(43) Pub. Date: Jun. 21, 2012

(54) PROCESS FOR THE AUTOMATIC CONTROL OF A RESPIRATOR

(75) Inventors: Marcus EGER, Lubeck (DE); Hans-Ullrich HANSMANN, Barnitz (DE); Tobias GLAW, Lubeck (DE); Frank SATTLER, Lubeck (DE)

(73) Assignee: DRAGER MEDICAL GMBH, Lubeck (DE)

(21) Appl. No.: 13/187,787

(22) Filed: Jul. 21, 2011

(30) Foreign Application Priority Data
Dec. 20, 2010 (DE) 10 2010 055 242.9

(51) Int. Cl. A61M 16/00

(52) U.S. Cl. 128/204.23

(57) ABSTRACT

A process for the automatic control of a respirator with a changeover between phases of respiration (inspiration and expiration), by a control unit checking a breathing activity signal for a threshold criterion. If the threshold criterion is met, a changeover is made and the control unit controls the fan of the respirator such that a pneumatic respiration variable (airway pressure, flow) is brought from an actual value to a preset target value for the new phase of respiration. The control unit further divides the change in the respiration variable, from the actual value to the target value, into a plurality of partial steps and checks the current breathing activity signal for the threshold criterion after each partial step. If this threshold criterion is no longer met, the state of operation of the respirator returns to the state before the last changeover, and otherwise, continues with the next partial step.

### CROSS REFERENCE TO RELATED APPLICATIONS

This application claims the benefit of priority under 35 U.S.C. §119 of German Patent Application DE 10 2010 055 242.9 filed Dec. 20, 2010, the entire contents of which are incorporated herein by reference.

### FIELD OF THE INVENTION

The present invention pertains to a process for the automatic control of a respirator (also known as a ventilator), in which a changeover is consecutively made between two phases of respiration (inspiration and expiration) from one into the other, by a control unit checking a picked-up breathing activity signal in a phase of respiration for a threshold criterion for changing over into the next phase of respiration, and if the threshold criterion is met, a changeover is made from one phase of respiration into the other, whereby the control unit is further set up to control the fan of the respirator when changing over into the new phase of respiration, such that a pneumatic respiration variable (airway pressure, flow) is brought from an actual value to a preset target value for the new phase of respiration.

### BACKGROUND OF THE INVENTION

Artificial respiration with respirators is aimed at relieving the respiratory muscles of a patient and at guaranteeing a sufficient supply of oxygen and elimination of carbon dioxide. This can happen by complete takeover of the breathing activity by the respirator or in an assisting process by partial takeover of breathing activity by the respirator, whereby in the latter assisting process, a present breathing activity of the patient is assisted or reinforced. For this, the respirators contain a fan for supplying breathing gas with a pressure, which is preset by a control unit. Furthermore, sensors are present, which detect pneumatic breathing signals in a time-dependent manner, for example, airway pressure, volume flow (flow) of the breathing gas and volume (which results from the integration of the flow), and forward these to the control unit.

In view of the rise in chronic lung diseases and the demand for an improved therapy, noninvasive breathing assistance with improved interaction of the patient and respirator is a decisive requirement of modern respirators. An essential object herein is to establish time-based synchronicity between the device-side assistance and the patient's own breathing activity. Spontaneously breathing patients were frequently sedated in the past to adjust the respiration correctly and to force synchronicity between patient and respirator. This procedure is no longer acceptable by today's knowledge since risks of lung damage caused by the respiration have to be dealt with.

For an improved synchronization between the breathing activity of the patient and the fan action, it is important to detect the beginning of inspiration and the beginning of expiration in the breathing activity of the patient early and reliably. The breathing phase detection is especially often incorrect or late in newborns and in Chronic Obstructive Pulmonary Disease (COPD) patients using conventional processes and leads to increased respiratory work until exhaustion.

For an artificial respiration which shall take the patient's breathing activity into consideration in an improved manner, it is known from DE 10 2007 062 214 B3 to pick up electromyographic signals, besides pneumatic breathing activity signals, by means of electrodes placed on the thorax and to derive electromyographic breathing activity signals (EMG signals) therefrom. These EMG signals are independent of the pneumatic breathing activity signals and therefore represent an independent source of information, which can be used to detect the beginning of inspiration and expiration. The EMG signals are, however, not infrequently superimposed by interferences, for example, the ECG signal of the heart, motion artifacts or so-called cross-talk (muscle activity that has nothing to do with the respiratory system of the patient).

For the last-mentioned reason, EMG signals cannot be used as the sole basis for the detection of the beginning of inspiration and expiration and the corresponding control of the respirator.

A triggering of breaths on the basis of EMG signals is described in U.S. Pat. No. 6,588,423 B1. Here, the EMG raw signal is preprocessed and is finally used for triggering an intensity indicator (root mean square) of the EMG signal, whereby a fixed threshold is used—related to one breath.

In practice, however, the preprocessed EMG signal is often more susceptible to interferences than pneumatic signals (pressure or volume flow). Such a susceptibility to interferences or volatility makes it more difficult to change over or trigger the breaths when using trigger thresholds, since too many breaths may be mistakenly triggered (so-called autotrigger) or may be triggered too late (so-called delayed or missed trigger).

Marking of the signals with interferences can be avoided by suitable filterings (e.g., by means of sliding averaging) of the signals, which would result in the major drawback of an additional signal delay for the intended use for changing over between phases of respiration, however.

### SUMMARY OF THE INVENTION

An object of the present invention is to provide a process for the automatic control of a respirator, which keeps the effects of incorrectly induced changeovers into the next phase of respiration, i.e., from inspiration into expiration or from expiration into inspiration as low as possible.

According to the present invention, provisions are made for the respiration variable to be guided by the fan to the target value not in one step, but in a plurality of consecutive partial steps, after triggering the changeover into the new phase of respiration. After each partial step, the then current breathing activity signal is checked for the threshold criterion responsible for the present changeover again, and, if this threshold criterion is then no longer met, a return into the state of operation of the respirator before the currently triggered changeover is automatically brought about. In other words, a complete changeover into the next phase of respiration is not performed immediately, but rather this is performed in partial steps, and after each partial step, the hypothesis is checked as to whether the threshold criterion, which triggered the beginning of the changeover, has indicated an actual change of phase of respiration or an interference. If the threshold criterion is no longer met after the partial step this is an indication that the meeting of the threshold criterion in the triggering of the first partial step and possibly of other previous partial steps was rather brought about by an interference, and the further course of the breathing activity signal is no longer consistent with the assumption that a changeover into the next phase of respiration should actually be triggered. An incorrectly triggered changeover may therefore be taken back again already after one or a few partial steps by the respirator automatically returning into the last phase of respiration before the triggering of the changeover. Consequently, an incorrectly triggered changeover is interrupted again already at an early stage and is returned into the correct, actually still current, last phase of respiration before the incorrectly triggered changeover.

Consequently, the effects of an incorrectly triggered changeover are considerably reduced since a full inspiration or full expiration will not incorrectly pass through, if a changeover has incorrectly been triggered by an interference. If the effects of incorrect changeovers are considerably reduced in this respect by the process according to the present invention, the process according to the present invention also makes it possible to preset the threshold criteria as more sensitive, since a certain increase in incorrectly met threshold criteria can be tolerated by interferences based on the markedly reduced effects of such incorrectly triggered changeovers.

The concept of this “incremental” changeover with the respective checking whether the breathing activity signal is still consistent with the hypothesis of an actual transition into the next phase of respiration is preferably applied to breathing activity signals that are not directly affected by the control of the respirator. The respirator controls the pneumatic variables of the breathing process, so that pneumatic breathing activity signals such as airway pressure and volume flow (flow) are directly affected by the respirator. Breathing activity signals, which are not of a pneumatic nature and in this respect are not directly affected by the respirator, are, for example, electromyographic signals, which are picked up by electrodes on the skin surface of the thorax. This is especially the case, since the electric activity of the respiratory muscles does not immediately react to a stroke of the fan. In pneumatic (or derived therefrom) signals, the time window for a possible return into the previous phase of respiration is limited based on principle, because, when the threshold criterion is met, the fan reacts rapidly and rapidly changes pneumatic breathing activity signals.

According to another aspect of the invention, a respirator is provided with a breathing gas source connected to a fan. This may be fan controlling for conveying breathing gas through connection lines to a patient and may also be a controllable respiratory gas pressure supply. A control unit controls the fan or the gas pressure supply. One or more sensors are connected to the control unit for picking up at least one pneumatic breathing activity signal. The sensors are connected to the control unit. The control unit controls the respirator according to the process including consecutively changing over between inspiration and expiration phases of respiration including controlling the fan of the respirator when changing over into a new phase of respiration, such that a pneumatic respiration variable is brought from an actual value to a preset target value for the new phase of respiration.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a graph with a thinly dashed curve representing the curve of the respiration variable set by the respirator, in the present example the curve of the volume flow (flow) preset by the respirator; and

FIG. 2 is a schematic view of a respirator according to the invention.

### DESCRIPTION OF THE PREFERRED EMBODIMENTS

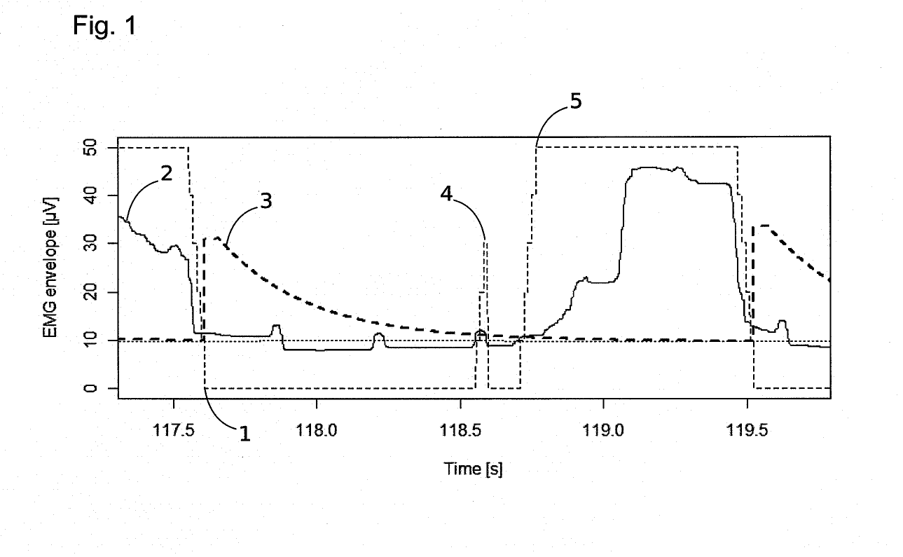

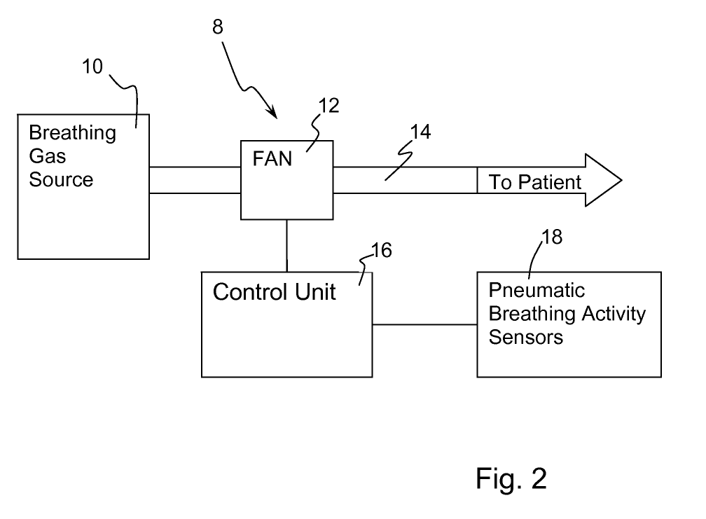

Referring to the drawings in particular, the present invention is described below based on an example in the figures, in which FIG. 1 shows the curve of the breathing activity signal, the curve of a threshold criterion and the curve of the guiding of the respiration variable by the respirator as functions of time. FIG. 2 shows a respirator 8 with a breathing gas source 10 connected to a fan 12 for conveying breathing gas through connection lines 14 to a patient. A control unit 16 controls the fan 12. Sensors 18 are connected to the control unit 16 for picking up at least one pneumatic breathing activity signal. The sensors 18 are connected to the control unit 16. The control unit controls the respirator for a consecutively made changeover between inspiration and expiration phases of respiration including controlling the fan of the respirator when changing over into a new phase of respiration, such that a pneumatic respiration variable is brought from an actual value to a preset target value for the new phase of respiration.

At first, FIG. 1 shows a preprocessed EMG signal. Such a preprocessing of the EMG raw signal takes place in the known manner in such a way that the EMG raw signal is freed from interference signals (e.g., ECG, motion artifacts, humming) and finally an envelope detection is performed. An envelope detection may be done, for example, by “rectification” and subsequent low-pass filtering, whereby the “rectification” is done by an operation imaging the quantity (e.g., by squaring or pure quantity formation). After a low-pass filtering, the envelope is obtained, i.e., the curve enveloping the signal pattern of the raw signal. A preferred realization of envelope detection is the formation of the so-called RMS (Root Mean Square) over the length of a sliding time window. The concept of EMG amplitude estimation, which is defined by the term “envelope detection,” is described in detail in Merlett, R., Parker P. A.: Electromyography. Physiology, Engineering, and Noninvasive Applications. IEEE Press, Wiley Interscience, 2004, starting from chapter 6.4 or pages 139 ff (the contents of which are incorporated herein by reference).

This preprocessed EMG signal is designated as 2 in FIG. 1. The dashed curve designated by 3 shows a threshold, a time-dependent dynamic threshold in this case. Basically, the embodiment of the threshold is not important for the present invention, the present invention can advantageously also be applied with constant thresholds. However, in the present example, a dynamic threshold is also suitable for detecting the next inspiration, which, at the beginning of an expiration, begins at first with a relatively high value for a preset period of time and is then lowered to a target value, which will be reached at the expected point in time of the end of expiration; the expected endpoint of expiration may be estimated, for example, from the duration of the past expiration phases. The preset period of time, for which the threshold is maintained at a relatively high constant value after the beginning of expiration, will be selected, such that it is markedly shorter than all expected expiration phases, i.e., the probability that a new inspiration already begins during the period of time should be very low; such a period of time may be, for example, 200 msec. The target value of the threshold can be derived, e.g., from the maximum values and minimum values of the signals u_{pneumatic}^{max} and u_{pneumatic}^{min} and u_{non-pneumatic}^{max} and u_{non-pneumatic}^{min} in previous phases of respiration.

Inversely, the threshold begins for the detection of the next expiration when an inspiration begins with a low value for a preset period of time and is then raised to a preset target value, which will be reached at the expected point in time of the end of the inspiration. The expected endpoint of the inspiration can in turn be estimated from the duration of the past inspiration phases and the target values can be derived from the maximum values and minimum values of the signals u_{pneumatic}^{max} and u_{pneumatic}^{min} and u_{non-pneumatic}^{max} and u_{non-pneumatic}^{min} in previous phases of respiration.

The idea, on which such dynamic thresholds are based, is that, right after the beginning of a phase of respiration, the probability is very low that a change into the next phase of respiration could already take place again within the preset short period of time. Therefore, it is possible to work with set thresholds at the beginning of a phase of respiration such that an incorrect changeover is almost ruled out even in case of great interferences, while, after the preset period of time, the threshold is brought to a target value, which will be reached at the expected end of the phase of respiration, such that the beginning of the next phase of respiration can be detected with high sensitivity. The expected value may correspond to the value that is suitable for a sensitive constant threshold. In this respect, towards the end of the phase of respiration, a sensitive triggering is guaranteed, at the beginning of which, however, a mistriggering due to interferences is suppressed.

The view of FIG. 1 begins at the left end with the rest of an inspiration phase, i.e., the volume flow (thinly dotted line) is set to a high constant value. The EMG signal 2 already begins to drop towards the end of this inspiration phase and drops at the end sharply to a base value. At the end of this sharp drop, the threshold criterion is met for the triggering of the changeover into expiration, whereupon the volume flow is reduced by the respirator from the high constant value. This takes place now not in a single step, but in a plurality of partial steps, whereby a brief delay is provided after each partial step, such that a stair-like course of the volume flow can be observed. After each partial step or on each step of the stair-like course, the threshold criterion which triggered the changeover is checked again with the current breathing activity signal. Insofar as the breathing activity signal meets, furthermore, the threshold criterion triggering the changeover, the partial step sequence is continued. In the changeover into expiration shown on the left in FIG. 1, this is the case because the volume flow signal takes a gradually descending course to the base value, at which it then remains for a longer time.

In the expiration phase starting from approximately 117.6 sec, the EMG signal remains low for the most part, only small signal increases (artifacts due to the ECG signal) can be detected, which, however, at first remain markedly below the dynamic threshold 3. Towards the end of the expiration phase, such an artifact appears again in the area provided with the reference number 4. At this time, the dynamic threshold is, however, already relatively low, since the duration of the present expiration phase is already close to the expected value for expiration phases. Hence, the EMG signal, due to the artifact appearing in the area of reference number 4, meets the threshold criterion for changing over into the next phase of respiration (inspiration). As a result the respirator initiates the raising of the value of the respiration variable. This raising of the volume flow takes place in turn in partial steps. After each partial step of the stair-like course of the increase in the volume flow, the threshold criterion triggering this changeover is again checked with the then present breathing activity signal and the increase is continued only if the threshold criterion is still met. In the area of artifact 4, this is the case for three partial steps, i.e., it will pass through three partial steps or stages of the increase. When the threshold criterion triggering this changeover into inspiration is then checked again with the then present breathing activity signal, it is determined, however, that the threshold criterion is no longer met, since the artifact in area 4 lasts for only a relatively short time and already dropped so far again after the third partial step of initiating the inspiration that the threshold criterion for the changeover into inspiration is no longer met. The changeover into inspiration is thereupon interrupted and the respirator is set back into the state that was present before initiating the inspiration in area 4, i.e., in the expiration state. This expiration state then still lasts for a short time.

After that, the EMG signal begins to show a continuous increase in activity. At first, this leads to the threshold criterion being met again for changing over into inspiration. Thereupon, the setting of the respiration variable volume flow to the inspiration state begins in a plurality of partial steps (in the example shown there are five partial steps). Since the EMG signal is increased steadily at this point in time, at the beginning of a true inspiration, the step-like increase in the volume flow is continued without return and the changeover into inspiration is steadily maintained. The process then continues with inspiration in the above-described manner until the changeover into the next expiration phase takes place on the right in FIG. 1.

The constant phase after each partial step may be, for example, approximately 50 msec. In this time, the triggering threshold criterion is checked, and, if the condition for assuming the transition into the next phase of respiration is no longer met, a return is made to the respiration state of the respirator valid before the initiation of the changeover. The reason why the threshold criterion is no longer met may be both a change in the breathing activity signal and a change in a dynamic threshold that occurred after triggering the first partial step; however, the latter case is of lesser importance since dynamic thresholds change very slowly in periods of the partial steps of the changeover, especially towards the end of a phase of respiration. However, if the threshold criterion continues to be met after a partial step, the next partial step to the next stage takes place. If the last stage is reached, this is synonymous with a complete change into the next phase of respiration.

The number of partial steps is preferably two to ten. The time for the changeover from airway pressure or flow to the preset target value is typically selected to be in a range of 100 msec to 300 msec, preferably about 200 msec. This corresponds to a period of about 20 msec to 100 msec for the individual partial step.

The partial steps may be uniform in the sense that the increment of the change in respiration variable is equal for all partial steps. However, it is preferred that the increment of the first partial step be smaller than the at least one next partial step. Also, the increment from the first to the last partial step may increase from step to step or after a plurality of partial steps. A method of proceeding with incremental increasing has the advantage that at first smaller increments are first selected in the first partial step or first partial steps, because, shortly after the beginning of the changeover, the probability is highest that the triggering of the changeover was caused by an interference and that a return to the last respiration state is necessary. After partial steps have already taken place, it is always unlikely that the by and large already performed changeover was triggered by an interference, since the threshold criterion has now already proven to be met over a plurality of past partial steps.

Provisions may be made that the change in set point in each partial step is limited to a selectable peak of the rate of change. This is actually also preferable, so that the sequence of the partial steps does not represent a true step function with vertical or almost vertical increases or drops, but rather a continuous increase takes place in each partial step. With corresponding selection of the peak value of the rate of change, a continuous linear increase may result in each partial step, such that the sequence of the partial steps then produces overall a ramp-like increase from the actual value to the set point. The return after interruption of the begun changeover into the state of the previous phase of respiration should, of course, take place as fast as possible, such that no limitation of the rate of change is anticipated with this.

The limitation of the rate of change is preferably applied to the set point curve of the respiration variable, but can also be realized, e.g., by a short-term change in control properties of the respirator. Preferably, the limitation of the rate of change vₘₐₓ and the width τ and height hᵢ of the ith partial step is adapted, e.g., by the formula vₘₐₓ=hᵢ/τ.

Instead of a limitation of the rate of change of the set point curve, the set point curve may, as an alternative, be subject to a low-pass filtering, which leads to a smoothing of the step-like signal. Preferably, the cutoff frequency f of the low-pass filter is adapted to the width of the partial steps τ, e.g., by the formula f=1/(2πτ).

While specific embodiments of the invention have been shown and described in detail to illustrate the application of the principles of the invention, it will be understood that the invention may be embodied otherwise without departing from such principles.

What is claimed is:

1. A process for the automatic control of a respirator, the process comprising the steps of:
using a control unit for a consecutively made changeover between inspiration and expiration phases of respiration from one into the other;
checking a picked-up breathing activity signal in a phase of respiration for a threshold criterion for changing over into a next phase of respiration;
changing over from one phase of respiration into the other, if the threshold criterion is met;
controlling the fan of the respirator when changing over into a new phase of respiration, such that a pneumatic respiration variable is brought from an actual value to a preset target value for the new phase of respiration;
dividing the change in the respiration variable from the actual value to the target value into a plurality of partial steps when changing over the phase of respiration;
checking a current breathing activity signal for the threshold criterion responsible for triggering the changeover after each partial step;
returning automatically into the state of operation of the respirator before a last changeover if the threshold criterion is no longer met; and
continuing with the next partial step if the threshold criterion continues to be met.

2. A process in accordance with claim 1, wherein increments of the partial steps are selected from set point to actual value, such that the increment of a first partial step is smaller than the at least one next partial step.

3. A process in accordance with claim 2, wherein the increments of the partial steps from set point to actual value are selected, such that the increment increases from partial step to partial step or in a plurality of stages from first to last partial step.

4. A process in accordance with claim 1, wherein a change rate of the respiration variable is limited in each partial step to a selectable peak value.

5. A process in accordance with claim 1, wherein a set point curve provided for guiding the respiration variable from actual value to set point is subject to low-pass filtering.

6. A process in accordance with claim 5, wherein a cutoff frequency f of a low-pass filter providing the low-pass filtering is adapted to a width of the partial steps τ.

7. A process in accordance with claim 6, wherein the cutoff frequency f of the low-pass filter providing the low-pass filtering is adapted to a width of the partial steps τ by the formula f=1/(2πτ).

8. A process in accordance with claim 6, wherein the pneumatic respiration variable is one or more of airway pressure and airway flow.

9. A process for the automatic control of a respirator, the process comprising the steps of:
providing a breathing gas source;
providing a fan for conveying breathing gas through connection lines;
providing a control unit controlling the fan;
providing sensors for picking up at least one pneumatic breathing activity signal;
connecting the sensors to the control unit;
using the control unit for a consecutively made changeover between inspiration and expiration phases of respiration from one into the other including:
checking a picked-up breathing activity signal in a phase of respiration for a threshold criterion for changing over into a next phase of respiration;
changing over into the next phase of respiration, if the threshold criterion is met;
controlling the fan of the respirator when changing over into the next phase of respiration, such that a pneumatic respiration variable is brought from an actual value to a preset target value for the next phase of respiration;
dividing the change in the respiration variable from the actual value to the target value into a plurality of partial steps;
checking a current breathing activity signal for the threshold criterion responsible for triggering the changeover after each partial step;
returning automatically into the phase of respiration before the last changeover if the current breathing activity signal no longer meets the threshold criterion; and
continuing with the next partial step if the threshold criterion continues to be met.

10. A process in accordance with claim 9, wherein increments of the partial steps are selected from set point to actual value, such that an increment of a first partial step is smaller than an increment of at least one next partial step and the increment increases from partial step to partial step or in a plurality of stages from first to last partial step.

11. A process in accordance with claim 9, wherein a change rate of the respiration variable is limited in each partial step to a selectable peak value.

12. A process in accordance with claim 9, wherein a set point curve provided for guiding the respiration variable from actual value to set point is subject to low-pass filtering.

13. A process in accordance with claim 12, wherein a cutoff frequency f of a low-pass filter providing the low-pass filtering is adapted to a width of the partial steps τ.

14. A process in accordance with claim 13, wherein the cutoff frequency f of the low-pass filter providing the low-pass filtering is adapted to a width of the partial steps τ by the formula f=1/(2πτ).

15. A respirator comprising:
a breathing gas source;
a fan for conveying breathing gas through connection lines;
a control unit controlling the fan; and
sensors for picking up at least one pneumatic breathing activity signal, the sensors being connected to the control unit, the control unit controlling the respirator for a consecutively made changeover between inspiration and expiration phases of respiration, the controlling of the respirator comprising:
checking a picked-up breathing activity signal in a phase of respiration for a threshold criterion for changing over into a next phase of respiration;
changing over from one phase of respiration into the other, if the threshold criterion is met;
controlling the fan of the respirator when changing over into a new phase of respiration, such that a pneumatic respiration variable is brought from an actual value to a preset target value for the new phase of respiration;
dividing the change in the respiration variable from the actual value to the target value into a plurality of partial steps when changing over the phase of respiration;
checking the current breathing activity signal for the threshold criterion responsible for triggering the changeover after each partial step;
returning automatically into the state of operation of the respirator before the last changeover if the threshold criterion is no longer met; and
continuing with the next partial step if the threshold criterion continues to be met.

16. A respirator in accordance with claim 15, wherein increments of the partial steps are selected from set point to actual value, such that the increment of a first partial step is smaller than the at least one next partial step.

17. A respirator in accordance with claim 16, wherein the increments of the partial steps from set point to actual value are selected, such that the increment increases from partial step to partial step or in a plurality of stages from first to last partial step.

18. A respirator in accordance with claim 16, wherein a change rate of the respiration variable is limited in each partial step to a selectable peak value.

19. A respirator in accordance with claim 16, further comprising a low-pass filter wherein:
a set point curve provided for guiding the respiration variable from actual value to set point is subject to low-pass filtering with the low-pass filter; and
a cutoff frequency f of the low-pass filter is adapted to a width of the partial steps.

20. A respirator in accordance with claim 19, wherein the cutoff frequency f of the low-pass filter providing the low-pass filtering is adapted to a width of the partial steps τ by the formula f=1/(2πτ).
